SEQUENCE LISTING (11 sequences)

SEQ ID NO: 1 - protein, 3 aa, Artificial
  Keratin-derived
EEE

SEQ ID NO: 2 - protein, 4 aa, Artificial
  Keratin-derived
YEEE

SEQ ID NO: 3 - protein, 4 aa, Artificial
  Keratin-derived
EEEI

SEQ ID NO: 4 - protein, 5 aa, Artificial
  Keratin-derived
YEEEI

SEQ ID NO: 5 - protein, 6 aa, Artificial
  Keratin-derived
YEEEIR

SEQ ID NO: 6 - protein, 7 aa, Artificial
  Keratin-derived
YEEEIRV

SEQ ID NO: 7 - protein, 8 aa, Artificial
  Keratin-derived
YEEEIRVL

SEQ ID NO: 8 - protein, 9 aa, Artificial
  Keratin-derived
LYEEEIRVL

SEQ ID NO: 9 - protein, 5 aa, Artificial
  Keratin-derived
EEEIR

SEQ ID NO: 10 - protein, 6 aa, Artificial
  Keratin-derived
EEEIRV

SEQ ID NO: 11 - protein, 7 aa, Artificial
  Keratin-derived
EEEIRVL

(19) United States
(12) Patent Application Publication
(10) Pub. No.: US 2018/0353418 A1
(43) Pub. Date: Dec. 13, 2018

(54) HIGH-PERFORMANCE HAIR TREATMENT AGENTS WITH ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/359,028

(22) Filed: Nov. 22, 2016

(30) Foreign Application Priority Data
Dec. 1, 2015 (DE) 10 2015 223 841.5

(51) Int. Cl. A61K 8/73; A61K 8/46; A61K 8/49; A61K 8/365; A61K 8/36; A61Q 5/02; A61Q 5/12

(52) U.S. Cl. CPC A61K 8/737; A61K 8/731; A61K 8/463; A61K 8/4953; A61K 2800/5426; A61K 8/361; A61Q 5/02; A61Q 5/12; A61K 2800/596; A61K 8/365

(57) ABSTRACT

Hair treatment agents containing at least one anionic surfactant, at least one amphoteric and/or nonionic surfactant, at least one cationic polysaccharide polymer, hexetidine, at least one organic acid and at least one lipid component reduce or prevent the washing-out of color from colored hair.

### CROSS-REFERENCE TO RELATED APPLICATION

This application claims priority to German Patent Application No. 10 2015 223 841.5, filed Dec. 1, 2015, which is incorporated herein by reference in its entirety.

### TECHNICAL FIELD

This disclosure relates to hair treatment agents, in particular shampoos and so-called conditioners, containing an active substance combination for gentle and effective hair care.

### BACKGROUND

Care products having an effect that is as long-lasting as possible are becoming increasingly important, not least due to the severe stressing of hair that is brought about for example by coloring or perming and also by cleaning the hair with shampoos, and by environmental pollution. Such care agents influence the natural structure and the properties of the hair. For instance, the wet and dry combability of the hair and also the hold and body of the hair can be optimized following such care treatments, or the hair can be protected against increased splitting.

It has thus long been customary to subject hair to a special aftertreatment. During this, the hair is treated, for example in the form of conditioning, with specific active substances, for example quaternary ammonium salts or specific polymers. Depending on the formulation, this treatment improves the combability, the hold and the body of the hair and reduces the rate of splitting.

Multifunctional cosmetic products are also known in the prior art. These include in particular the so-called “2 in 1” shampoos, which not only clean but also condition the hair. Such products are greatly appreciated by consumers since, due to the product performance thereof, they enable the omission of at least one method step, for example the conditioning using a conventional hair conditioner.

Products for changing the natural color of the hair also play a prominent role in hair cosmetics. A distinction is made between permanent, semi-permanent or temporary coloring systems based on chemical and/or natural dyes. However, the hair colors artificially produced by permanent, semi-permanent or temporary coloring systems have the disadvantage that they may change in an undesirable manner, for example during or after hair cleaning.

An “undesirable change” will be understood to mean fading or bleeding or also the loss of color brilliance of the shade achieved by the respective coloring on the hair. Environmental influences and/or the effects of sunlight may further intensify these changes.

The use of divalent metal salts in hair coloring agents to improve the longevity and thus the fastness of the coloring is known from EP 2438900 A1.

There is still a need to provide active substances or active substance combinations for hair treatment agents having good care properties, which moreover strengthen the adhesion of dyes to the hair fibers and thus obtain the fastness of the artificially produced hair color, and to further develop hair treatment agents in this regard.

However, it has been observed that the water hardness can have a negative effect on the care properties of hair treatment agents, so that the care performance of one and the same product is either too low or too high depending on water quality.

Too high a care performance, or “excessive care” (occurrence of a so-called build-up effect), of the hair will be understood to mean especially a greasy feel to the hair, little volume to the hair and/or a lank, bedraggled appearance of the hair, whereas damaged hair (brittle, dull hair that is susceptible to splitting) is an indication of too low a care performance.

### BRIEF SUMMARY

Hair treatment agents and methods are provided herein. In an embodiment, a hair treatment agent includes:

a) at least one anionic surfactant,
b) at least one cationic surfactant,
c) at least one cationic polysaccharide polymer,
d) hexetidine,
e) at least one organic acid, and
f) at least one lipid component.

In another embodiment, a method for treating hair includes applying a hair treatment agent to dry or moist hair, wherein the hair treatment agent includes:

a) at least one anionic surfactant,
b) at least one cationic surfactant,
c) at least one cationic polysaccharide polymer,
d) hexetidine,
e) at least one organic acid, and
f) at least one lipid component,
where the hair treatment is agent is left on the hair for a period of about 30 to about 300 seconds and then rinsed off.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The object of the present application was therefore to provide skin-friendly hair treatment agents having a good care effect, which have excellent foaming properties and good rinsability with water and which in addition reduce or prevent the washing-out of color from colored hair.

The aim was for the hair treatment agents having a care effect to have a conditioning performance that remains constant regardless of the water quality and in particular not to weigh down fine hair and/or damaged hair and to strengthen the structure of the hair and thus protect it against splitting and breaking and to improve the combability and feel. The aim was also for the agents to have a germ-reducing effect.

It has now been found that a combination of specific ingredients has a particularly positive effect on colored hair treated therewith and on the hair follicles.

A first subject matter as contemplated herein relates to hair treatment agents containing a) at least one anionic surfactant,
b) at least one amphoteric and/or nonionic surfactant,
c) at least one cationic polysaccharide polymer,
d) hexetidine,
e) at least one organic acid,
f) at least one lipid component.

In the context as contemplated herein, hair treatment agents are for example hair shampoos, leave-in hair conditioners, conditioning shampoos, hairsprays, rinse-out hair conditioners, intensive hair conditioners, hair masks, hair tonics, perm fixing solutions, hair coloring shampoos, hair coloring agents, hair setting agents, hair smoothing agents, hair styling preparations, blow-drying lotions, mousses, hair gels, hair waxes or combinations thereof. In view of the fact that men in particular are often reluctant to use multiple different agents and/or multiple application steps, preference is given to those agents which a man already uses. Preferred agents are therefore shampoos, conditioners or hair tonics.

The hair treatment agents contain at least one anionic surfactant.

Suitable anionic surfactants and emulsifiers for the compositions as contemplated herein are any anionic surface-active substances suitable for use on the human body. These are characterized by a water-solubilizing anionic group such as for example a carboxylate, sulfate, sulfonate or phosphate group and a lipophilic alkyl group having around 8 to 30 C atoms. In addition, glycol or polyglycol ether groups, ester, ether and amide groups and also hydroxyl groups may also be contained in the molecule. The following are examples of suitable anionic surfactants and emulsifiers, in each case in the form of the sodium, potassium and ammonium salts and the mono-, di- and trialkanolammonium salts having 2 to 4 C atoms in the alkanol group
linear and branched fatty acids having 8 to 30 C atoms (soaps),
ether carboxylic acids of formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
acyl glutamates of formula (T-I)

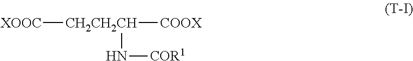

in which R¹CO represents a linear or branched acyl radical having 6 to 22 carbon atoms and 0, 1, 2 or 3 double bonds and X represents hydrogen, an alkali and/or alkaline earth metal, ammonium, alkylammonium, alkanolammonium or glucammonium, for example acyl glutamates derived from fatty acids having 6 to 22, preferably 12 to 18 carbon atoms, such as for example C_{12/14} or C_{12/18} coco fatty acid, lauric acid, myristic acid, palmitic acid and/or stearic acid, in particular sodium N-cocoyl-L-glutamate and sodium N-stearoyl-L-glutamate esters of a hydroxy-substituted di- or tricarboxylic acid of general formula (T-II)

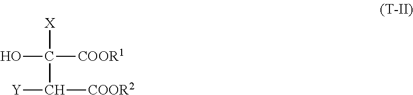

in which X═H or a —CH₂COOR group, Y═H or —OH, on condition that Y═H if X═—CH₂COOR, R, R¹ and R² independently of one another are a hydrogen atom, an alkali or alkaline earth metal cation, an ammonium group, the cation of an ammonium-organic base or a radical Z which is derived from a polyhydroxylated organic compound selected from the group consisting of etherified (C₆-C₁₈) alkylpolysaccharides having 1 to 6 monomeric saccharide units and/or the etherified aliphatic (C₆-C₁₆) hydroxyalkyl polyols having 2 to 16 hydroxyl radicals, with the proviso that at least one of the groups R, R¹ or R² is a radical Z,
esters of sulfosuccinic acid or of sulfosuccinates of general formula (T-III)

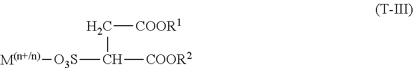

in which M^{(n+/n)} for n=1 is a hydrogen atom, an alkali metal cation, an ammonium group or the cation of an ammonium-organic base and for n=2 is an alkaline earth metal cation, and R¹ and R² independently of one another are a hydrogen atom, an alkali or alkaline earth metal cation, an ammonium group, the cation of an ammonium-organic base or a radical Z which is derived from a polyhydroxylated organic compound selected from the group consisting of etherified (C₆-C₁₈) alkylpolysaccharides having 1 to 6 monomeric saccharide units and/or the etherified aliphatic (C₆-C₁₆) hydroxyalkyl polyols having 2 to 16 hydroxyl radicals, with the proviso that at least one of the groups R¹ or R² is a radical Z,
sulfosuccinic acid monoalkyl and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
alkyl sulfates and alkyl polyglycol ether sulfates of formula R—(O—CH₂—CH₂)ₓ—OSO₃H, in which R is a preferably linear alkyl group having 8 to 30 C atoms and x=0 or 1 to 12,
mixed surface-active hydroxysulfonates according to DE-A-37 25 030,
esters of tartaric acid and citric acid with alcohols which are addition products of around 2-15 molecules of ethylene oxide and/or propylene oxide onto C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants and emulsifiers are acyl glutamates, acyl isethionates, acyl sarcosinates and acyl taurates, each with a linear or branched acyl radical having 6 to 22 carbon atoms and 0, 1, 2 or 3 double bonds, which in particularly preferred embodiments are selected from an octanoyl, decanoyl, lauroyl, myristoyl, palmitoyl and stearoyl radical, esters of tartaric acid, citric acid or succinic acid, or the salts of said acids with alkylated glucose, in particular the products having the INCI name Disodium Coco-Glucoside Citrate, Sodium Coco-Glucoside Tartrate and Disodium Coco-Glucoside Sulfosuccinate, alkyl polyglycol ether sulfates and ether carboxylic acids having 8 to 18 C atoms in the alkyl group and up to 12 ethoxy groups in the molecule, sulfosuccinic acid monoalkyl and dialkyl esters having 8 to 18 C atoms in the alkyl group and sulfosuccinic acid monoalkyl polyoxyethyl esters having 8 to 18 C atoms in the alkyl group and 1 to 6 ethoxy groups.

Further preferred anionic surfactants are alkyl sulfates, alkyl polyglycol ether sulfates and ether carboxylic acid salts having 10 to 18 C atoms in the alkyl group and up to 12 glycol ether groups in the molecule and sulfosuccinic acid monoalkyl and dialkyl esters having 8 to 18 C atoms in the alkyl group and sulfosuccinic acid monoalkyl polyoxyethyl esters having 8 to 18 C atoms in the alkyl group and 1 to 6 oxyethyl groups.

Particularly preferred anionic surfactants are the alkali or ammonium salts of lauryl ether sulfate with a degree of ethoxylation of 2 to 4 EO.

Preferred hair treatment agents contain, based on their weight, about 0.5 to about 20% by weight, preferably about 0.75 to about 15% by weight, more preferably about 1 to about 12% by weight and in particular about 2 to about 10% by weight anionic surfactant(s).

Particularly preferred hair treatment agents as contemplated herein are characterized in that they contain, based on their weight, about 0.5 to about 20% by weight, preferably about 0.75 to about 15% by weight, more preferably about 1 to about 12% by weight and in particular about 2 to about 10% by weight alkyl (ether) sulfates of general formula R—(OCH₂—CH₂)ₙ—OSO₃X, in which R is a straight-chain or branched, saturated or unsaturated alkyl group having 8 to 24 C atoms, n is the number 0 or 1 to 12, and X is an alkali, alkaline earth, ammonium or alkanolamine ion.

The hair treatment agents as contemplated herein contain at least one amphoteric surfactant and/or at least one nonionic surfactant. Amphoteric surfactants or zwitterionic surfactants are surfactants which have both a negatively charged and a positively charged functional group.

Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyl-dimethylammonium glycinate, and 2-alkyl-3-carboxymethyl-3-hydroxyethylimidazolines having in each case 8 to 18 C atoms in the alkyl or acyl group, and also cocoacylaminoethyl hydroxyethyl carboxymethyl glycinate. A preferred zwitterionic surfactant is the fatty acid amide derivative known under the INCI name Cocamidopropyl Betaine.

Further examples of suitable ampholytic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids having in each case around 8 to 24 C atoms in the alkyl group. Particularly preferred ampholytic surfactants are N-cocoalkylaminopropionate, cocoacylaminoethylaminopropionate and C₁₂-C₁₈ acyl sarcosine.

Preferred hair treatment agents as contemplated herein contain, based on their weight, about 0.3 to about 10% by weight, preferably about 0.5 to about 8% by weight, more preferably about 0.75 to about 6% by weight and in particular about 1 to about 5% by weight amphoteric surfactant(s).

Particularly preferred hair treatment agents as contemplated herein are characterized in that they contain amphoteric surfactants from the following groups
N-alkyl glycines,
N-alkylpropionic acids,
N-alkylaminobutyric acids,
N-alkyliminodipropionic acids,
N-hydroxyethyl-N-alkylamidopropyl glycines,
N-alkyl taurines,
N-alkyl sarcosines,
2-alkylaminopropionic acids having in each case around 8 to 24 C atoms in the alkyl group,
alkylaminoacetic acids having in each case around 8 to 24 C atoms in the alkyl group,
N-cocoalkylaminopropionate,
cocoacylaminoethylaminopropionate,
C₁₂-C₁₈ acyl sarcosine,
N-alkyl-N,N-dimethylammonium glycinates, for example cocoalkyldimethylammonium glycinate,
N-acylaminopropyl-N,N-dimethylammonium glycinates, for example cocoacylaminopropyl-dimethylammonium glycinate,
2-alkyl-3-carboxymethyl-3-hydroxyethylimidazolines having in each case 8 to 18 C atoms in the alkyl or acyl group,
cocoacylaminoethyl hydroxyethyl carboxymethyl glycinate,
the compounds known under the INCI name Cocamidopropyl Betaine,
the compounds known under the INCI name Disodium Cocoamphodiacetate,
   wherein preferred agents contain the amphoteric surfactant(s) in amounts of about 0.3 to about 10% by weight, preferably about 0.5 to about 8% by weight, more preferably about 0.75 to about 6% by weight and in particular about 1 to about 5% by weight, in each case based on the total agent.

Particularly preferred hair treatment agents contain, as amphoteric surfactants, betaines of formula (Bet-I)

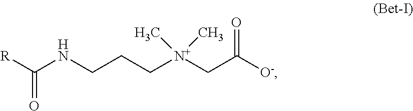

in which R represents a straight-chain or branched, saturated or mono- or polyunsaturated alkyl or alkenyl radical having 8 to 24 carbon atoms.

According to the INCI nomenclature, these surfactants are known as amidopropyl betaines, preference being given to those representatives which are derived from coco fatty acids and which are known as Cocamidopropyl Betaines. As contemplated herein, use is particularly preferably made of surfactants of formula (Bet-I) which are a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N+(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻

With particular preference, surfactants of formula (Bet-I) are used within relatively narrow amount ranges. Preference is given here to hair treatment agents as contemplated herein which contain, based on their weight, about 0.25 to about 8% by weight, more preferably about 0.5 to about 7% by weight, more preferably about 0.75 to about 6.5% by weight and in particular about 1 to about 5.5% by weight surfactant(s) of formula (Bet-I).

In addition to the amphoteric surfactant(s) of formula (Bet-I) or in place thereof, the hair treatment agents as contemplated herein may particularly preferably contain, as amphoteric surfactants, betaines of formula (Bet-II)

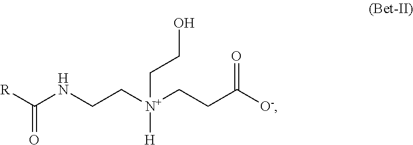

in which R represents a straight-chain or branched, saturated or mono- or polyunsaturated alkyl or alkenyl radical having 8 to 24 carbon atoms.

According to the INCI nomenclature, these surfactants are known as Amphoacetates, preference being given to those representatives which are derived from coco fatty acids and which are known as Cocoamphoacetates.

For production reasons, surfactants of this type always also contain betaines of formula (Bet-IIa)

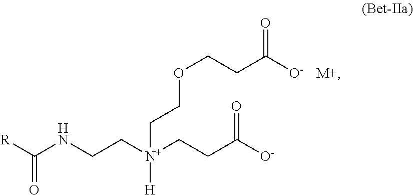

in which R represents a straight-chain or branched, saturated or mono- or polyunsaturated alkyl or alkenyl radical having 8 to 24 carbon atoms and M represents a cation.

According to the INCI nomenclature, these surfactants are known as Amphodiacetates, preference being given to those representatives which are derived from coco fatty acids and which are known as Cocoamphodiacetates.

As contemplated herein, use is particularly preferably made of surfactants of formula (Bet-II) which are a mixture of the following representatives:

H₃C—(CH₂)₇—C(O)—NH—(CH₂)₂NH⁺(CH₂CH₂OH)CH₂CH₂COO⁻

H₃C—(CH₂)₉—C(O)—NH—(CH₂)₂NH⁺(CH₂CH₂OH)CH₂CH₂COO⁻

H₃C—(CH₂)₁₁—C(O)—NH—(CH₂)₂NH⁺(CH₂CH₂OH)CH₂CH₂COO⁻

H₃C—(CH₂)₁₃—C(O)—NH—(CH₂)₂NH⁺E(CH₂CH₂OH)CH₂CH₂COO⁻

H₃C—(CH₂)₁₅—C(O)—NH—(CH₂)₂NH⁺(CH₂CH₂OH)CH₂CH₂COO⁻

H₃C—(CH₂)₇—CH═CH—(CH₂)₇—C(O)—NH—(CH₂)₂NH⁺(CH₂CH₂OH)CH₂CH₂COO⁻

With particular preference, surfactants of formula (Bet-II) are used within relatively narrow amount ranges. Preference is given here to hair treatment agents as contemplated herein which contain, based on their weight, about 0.25 to about 8% by weight, more preferably about 0.5 to about 7% by weight, more preferably about 0.75 to about 6.5% by weight and in particular about 1 to about 5.5% by weight surfactant(s) of formula (Bet-II).

To sum up, preference is given to cosmetic agents as contemplated herein in which the radical R in formulae (Bet-I) and (Bet-II) is selected from

H₃C—(CH₂)₇— H₃C—(CH₂)₉— H₃C—(CH₂)₁₁— H₃C—(CH₂)₁₃— H₃C—(CH₂)₁₅—H₃C—(CH₂)₇—CH═CH—(CH₂)₇— or mixtures thereof.

The hair treatment agents may contain nonionic surfactant(s). If they are free of amphoteric surfactants, they mandatorily contain a nonionic surfactant.

Suitable nonionic surfactants include for example
addition products of 4 to 30 mol ethylene oxide and/or 0 to 5 mol propylene oxide onto linear fatty alcohols having 8 to 22 C atoms, onto fatty acids having 12 to 22 C atoms and onto alkyl phenols having 8 to 15 C atoms in the alkyl group,
ethylene oxide and polyglycerol addition products onto methyl glucoside fatty acid esters, fatty acid alkanolamides and fatty acid glucamides,
C₈-C₃₀ fatty acid monoesters and diesters of addition products of 1 to 30 mol ethylene oxide onto glycerol,
amine oxides,
sorbitan fatty acid esters and addition products of ethylene oxide onto sorbitan fatty acid esters, such as for example polysorbates,
fatty acid alkanolamides of the following general formula

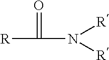

in which R is preferably a linear or branched, saturated or unsaturated alkyl or alkenyl radical having 8 to 24 carbon atoms and the radicals R′ are hydrogen or the group —(CH₂)ₙOH, in which n is the number 2 or 3, with the proviso that at least one of the radicals R′ represents the aforementioned radical —(CH₂)ₙOH,
sugar fatty acid esters and addition products of ethylene oxide onto sugar fatty acid esters,
addition products of ethylene oxide onto fatty acid alkanolamides and fatty amines, and/or
alkyl (oligo)glucosides,
mixtures of alkyl (oligo)glucosides and fatty alcohols, for example the commercially available product Montanov® 68,
addition products of 5 to 60 mol ethylene oxide onto castor oil and hydrogenated castor oil,
partial esters of polyols having 3 to 6 carbon atoms with saturated fatty acids having 8 to 22 C atoms,
sterols. Sterols are understood to mean a group of steroids which carry a hydroxyl group on the third carbon atom of the steroid structure and are isolated both from animal tissue (zoosterols) and from plant fats (phytosterols). Examples of zoosterols are cholesterol and lanosterol. Examples of suitable phytosterols are ergosterol, stigmasterol and sitosterol. Sterols are also isolated from fungi and yeasts, these sterols being known as mycosterols.
phospholipids, including especially the glucose phospholipids which are obtained for example as lecithins or phosphatidylcholines from for example egg yolk or plant seeds (for example soybeans).

Suitable alkyl (oligo)glycosides may be selected from compounds of general formula RO-[G]ₓ, in which [G] is preferably derived from aldoses and/or ketoses having 5-6 carbon atoms, preferably from glucose.

The index number x denotes the degree of oligomerization (DP), that is to say the distribution of the mono- and oligoglycosides. The index number x preferably has a value in the range from 1 to 10, particularly preferably in the range from 1 to 3, wherein it need not be a whole number but may be a fraction which can be determined analytically.

Particularly preferred alkyl (oligo)glycosides have a degree of oligomerization between 1.2 and 1.5.

The radical R preferably represents at least one alkyl and/or alkenyl radical having 4 to 24 C atoms.

Particularly preferred alkyl (oligo)glycosides are the compounds known under the INCI names Caprylyl/Capryl Glucoside, Decyl Glucoside, Lauryl Glucoside and Coco Glucoside.

Suitable amine oxides may be selected from at least one compound of general formulae (A-I) or (A-II)

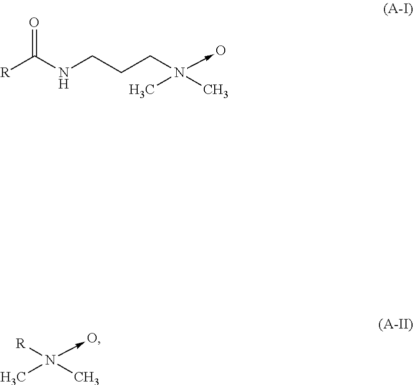

in which R in each case represents a straight-chain or branched, saturated or mono- or polyunsaturated alkyl or alkenyl radical having 6 to 24 carbon atoms, preferably having 8 to 18 carbon atoms.

Particular preference is given to the surfactants of the aforementioned formulae (A-I) or (A-II) which are known under the INCI names Cocamine Oxide, Lauramine Oxide and/or Cocamidopropylamine Oxide and are commercially available from various suppliers.

Suitable C₈-C₃₀ fatty acid monoesters and diesters of addition products of 1 to 30 mol ethylene oxide onto glycerol will preferably be understood to mean those under the INCI names PEG(1-10) Glyceryl Cocoate, in particular PEG-7 Glyceryl Cocoate.

It may also be advantageous to combine the ethoxylated fatty acid esters with further ethoxylated fatty acid esters. Such product mixtures are commercially available, for example under the name “Antil 200®” (INCI name: PEG-200 Hydrogenated Glyceryl Palmate, PEG-7 Glyceryl Cocoate) from the company Evonik.

Particularly preferred nonionic surfactants which may be contained in the hair treatment agents as contemplated herein are fatty acid alkanolamides, in particular the compounds known under the INCI names Cocamide MEA and/or Cocamide MIPA; alkyl (oligo)glucosides, in particular the compounds known under the INCI names Caprylyl/Capryl Glucoside, Decyl Glucoside, Lauryl Glucoside and/or Coco Glucoside; C₈-C₃₀ fatty acid monoesters and diesters of addition products of 1 to 30 mol ethylene oxide onto glycerol, in particular the compound known under the INCI name PEG-7 Glyceryl Cocoate; and/or addition products of 4 to 30 mol ethylene oxide and/or 0 to 5 mol propylene oxide onto linear fatty alcohols having 8 to 22 C atoms.

Particular preference is given to Cocamide MEA and/or PEG-7 Glyceryl Cocoate on account of their foam-stabilizing and moisturizing properties.

Hair treatment agents which are preferred as contemplated herein contain, based on their weight, about 0.3 to about 10% by weight, preferably about 0.5 to about 8% by weight, more preferably about 0.75 to about 6% by weight and in particular about 1 to about 5% by weight nonionic surfactant(s).

The hair treatment agents may contain cationic surfactant(s). As contemplated herein, use may be made of cationic surfactants of the quaternary ammonium compound, esterquat or amidoamine type. Preferred quaternary ammonium compounds are ammonium halides, in particular chlorides and bromides, such as alkyltrimethylammonium chlorides, dialkyldimethylammonium chlorides and trialkylmethylammonium chlorides. The long alkyl chains of said surfactants preferably have 10 to 18 carbon atoms, such as for example in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are the imidazolium compounds known under the INCI names Quaternium-27 and Quaternium-83.

Particularly preferred hair treatment agents as contemplated herein are characterized in that they contain as the cationic care substance, based on their weight, about 0.05 to about 7.5% by weight, preferably about 0.1 to about 5% by weight, particularly preferably about 0.2 to about 3.5% by weight and in particular about 0.25 to about 2.5% by weight cationic surfactant(s) from the group consisting of quaternary ammonium compounds and/or esterquats and/or amidoamines, preferred cationic surfactant(s) being selected from
alkyltrimethylammonium chlorides having preferably 10 to 18 carbon atoms in the alkyl radical and/or
dialkyldimethylammonium chlorides having preferably 10 to 18 carbon atoms in the alkyl radical and/or
trialkylmethylammonium chlorides having preferably 10 to 18 carbon atoms in the alkyl radical and/or
cetyltrimethylammonium chloride and/or
stearyltrimethylammonium chloride and/or
distearyldimethylammonium chloride and/or
lauryldimethylammonium chloride and/or
lauryldimethylbenzylammonium chloride and/or
tricetylmethylammonium chloride
Quaternium-27 and/or
Quaternium-83 and/or
N-methyl-N-(2-hydroxyethyl)-N,N-(ditalgacyloxyethyl)ammonium methosulfate and/or
N-methyl-N-(2-hydroxyethyl)-N,N-(distearoyloxyethyl)ammonium methosulfate and/or
N,N-dimethyl-N,N-distearoyloxyethylammonium chloride and/or
N,N-di-(2-hydroxyethyl)-N,N-(fatty acid ester ethyl)ammonium chloride.

The hair treatment agents contain at least one cationic polysaccharide polymer.

Cationic polysaccharide polymers increase the care performance of the hair treatment agents as contemplated herein (in particular the efficacy of the agents as contemplated herein against hair breakage). Suitable cationic polysaccharide polymers may be selected from cationic cellulose compounds and/or cationic guar derivatives.

Particularly preferred hair treatment agents as contemplated herein contain as cationic polysaccharide polymer(s) c), based on the weight of the agent, about 0.01 to about 3% by weight, preferably about 0.05 to about 2% by weight, more preferably about 0.1 to about 1.5% by weight and in particular about 0.15 to about 0.8% by weight of at least one polymer from the group consisting of cationic cellulose polymers and/or cationic guar derivatives.

As contemplated herein, cationic cellulose compounds are those which carry more than one permanent cationic charge in at least one side chain. Cellulose is constructed from beta-1,4-glycoside-linked D-glucopyranose units and forms unbranched, water-insoluble chains. The term “side chain” of a cellulose defines chemical substituents which bind to the cellulose backbone and are not part of the native cellulose, since they have been introduced subsequently for example by chemical synthesis.

Preference is given to quaternized cellulose polymers derived from hydroxy(C₂-C₄)alkylcelluloses, particularly preferably from hydroxyethylcelluloses.

Such polymers are known to the person skilled in the art and are commercially available from various companies. Particular preference is given to the cationic cellulose derivatives known under the INCI names Polyquaternium-4, Polyquaternium-10, Polyquaternium-24, Polyquaternium-67 and/or Polyquaternium-72. Very particular preference is given to Polyquaternium-10, Polyquaternium-24 and/or Polyquaternium-67 and preference is given in particular to Polyquaternium-10.

Preferred hair treatment agents as contemplated herein contain as cationic polysaccharide polymer(s) c), based on the weight of the agent, about 0.01 to about 3% by weight, preferably about 0.05 to about 2% by weight, more preferably about 0.1 to about 1.5% by weight and in particular about 0.15 to about 0.8% by weight of at least one polymer from the group consisting of Polyquaternium-4, Polyquaternium-10, Polyquaternium-24, Polyquaternium-67 and/or Polyquaternium-72.

Particularly preferred hair treatment agents as contemplated herein contain as cationic polysaccharide polymer(s) c), based on the weight of the agent, about 0.01 to about 3% by weight, preferably about 0.05 to about 2% by weight, more preferably about 0.1 to about 1.5% by weight and in particular about 0.15 to about 0.8% by weight Polyquaternium-10.

Suitable cationic guar derivatives in the context of the invention are cationic hydroxyalkyl guar derivatives, preferably cationic hydroxyethyltrimethylammonium guar and/or cationic hydroxypropyltrimethylammonium guar having average molecular weights of between about 100,000 and about 2,000,000 daltons. Particular preference is given to the cationic guar polymers known under the INCI name Guar Hydroxypropyltrimonium Chloride, having a molecular weight (weight-average) of between about 200,000 and about 1,600,000 daltons. The cationic charge density of said guar polymers is preferably at least 0.4 meq/g, more preferably at least 0.5 meq/g and in particular at least 0.6 meq/g. The nitrogen content thereof preferably lies in the range from about 1.1 to about 1.8% by weight (based on their total weight).

Cationic guar derivatives which are known under the INCI name Guar Hydroxypropyltrimonium Chloride are known to the person skilled in the art and are available from various suppliers for example under the trade names Cosmedia® Guar, N-Hance® and/or Jaguar®.

Particularly preferred hair treatment agents as contemplated herein contain as cationic polysaccharide polymer(s) c), based on the weight of the agent, about 0.01 to about 3% by weight, preferably 0.05 to 2% by weight, more preferably about 0.1 to about 1.5% by weight and in particular about 0.15 to about 0.8% by weight Guar Hydroxypropyltrimonium Chloride.

The hair treatment agents contain hexetidine. Hexetidine, also called 5-amino-1,3-bis(2-ethylhexal)hexahydro-5-methylpyrimidine or 1,3-bis(2-ethylhexyl)hexahydro-5-methylpyrimidin-5-amine is an antiseptic or disinfectant which is represented by the following formula:

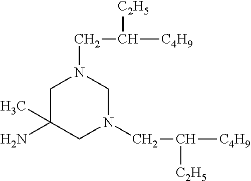

Hexetidine is used in the agents as contemplated herein preferably within specific relatively narrow amount ranges. Hair treatment agents which are preferred as contemplated herein contain, based on their weight, about 0.001 to about 0.3% by weight, preferably about 0.01 to about 0.2% by weight, more preferably about 0.025 to about 0.15% by weight and in particular about 0.05 to about 0.1% by weight hexetidine.

The hair treatment agents contain at least one organic acid.

Typical representatives of organic acids are aliphatic mono- and dicarboxylic acids such as for example acetic acid, propionic acid, oxalic acid and 1,3-propanedioic acid, as well as aromatic carboxylic acids such as for example benzoic acid. Further organic acids are for example hydroxycarboxylic acids such as glycolic acid, citric acid, tartaric acid, malic acid and lactic acid. Unsaturated mono- or dicarboxylic acids such as for example fumaric acid or α-ketocarboxylic acids such as for example pyruvic acid (2-oxopropionic acid) are also in accordance with the invention.

Hair treatment agents which are preferred as contemplated herein contain, based on their weight, about 0.001 to about 10% by weight, preferably about 0.01 to about 5% by weight, more preferably about 0.05 to about 3% by weight and in particular about 0.1 to about 2% by weight organic acid(s).

Particularly preferred hair treatment agents contain, based on their weight, about 0.001 to about 10% by weight, preferably about 0.01 to about 5% by weight, more preferably about 0.05 to about 3% by weight and in particular about 0.1 to about 2% by weight organic acid(s) from the group consisting of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-napthalenepentaacetic acid, malonaldehydic acid, 4-hydroxyphthalamidic acid, 1-pyrazolecarboxylic acid, gallic acid or propanetricarboxylic acid, glycolic acid, lactic acid, malic acid, tartaric acid or citric acid.

Among the aforementioned acids, some representatives are particularly preferred because they additionally give the hair treated with the agents as contemplated herein a pleasant shine. Extremely preferred hair treatment agents contain, based on their weight, about 0.001 to about 10% by weight, preferably about 0.01 to about 5% by weight, more preferably about 0.05 to about 3% by weight and in particular about 0.1 to about 2% by weight organic acid(s) from the group consisting of formic acid, oxalic acid, maleic acid, lactic acid, tartaric acid or citric acid.

The hair treatment agents contain at least one lipid component.

As contemplated herein, all fats and fat-like substances which preferably melt in the range from about 30 to about 150° C. can be used as lipid components. These include inter alia fats (triglycerides), mono- and diglycerides, waxes, fatty and wax alcohols, fatty acids, esters and/or ethers of fatty alcohols and fatty acids and also fatty acid amides or any mixtures of said substances.

Fats are understood to be triacylglycerols, that is to say the triple esters of fatty acids with glycerol. Among the triacylglycerols, those which contain saturated, unbranched and unsubstituted fatty acid residues are preferred as lipid components having a melting point between about 30 and about 150° C. These may also be mixed esters, that is to say triple esters of glycerol with various fatty acids. So-called hydrogenated fats and oils, which are obtained by partial hydrogenation, can also be used as contemplated herein and are particularly suitable as consistency regulators. Preference is given to hydrogenated plant fats and oils, for example hydrogenated castor oil, peanut oil, soybean oil, rape oil, rapeseed oil, cottonseed oil, soybean oil, sunflower oil, palm oil, palm kernel oil, linseed oil, almond oil, corn oil, olive oil, sesame oil, cocoa butter and coconut oil.

In one preferred embodiment, the lipid component is selected from the group of triple esters of glycerol with fatty acids. Particular preference is given to the triple esters of glycerol with C₁₈-C₆₀ fatty acids and in particular C₁₈-C₃₆ fatty acids. These include hydrogenated castor oil, a triple ester of glycerol and a hydroxystearic acid which is marketed for example under the name Cutina® HR, and also glycerol tristearate, glycerol tribehenate (for example Syncrowax® HRC), glycerol tripalmitate or the triglyceride mixtures known under the name Syncrowax® HGLC.

A combination of hydrogenated castor oil and C₁₈-C₃₆ fatty acid triglycerides (for example Syncrowax® HGLC) is also preferred as contemplated herein. By combining these triglycerides, the storage stability is increased. A combination of hydrogenated castor oil and long-chain C₂₀-C₄₀ fatty acids (for example Performacid® 350 Acid) has also proven to be advantageous as a lipid component.

Besides the triglycerides, it is also possible to use mono- and diglycerides and mixtures of glycerides as lipid components. The glyceride mixtures which are preferred as contemplated herein include the combination of Cutina® HR (hydrogenated castor oil) and Novata® AB (mixture of C₁₂-C₁₈ mono-, di- and triglycerides).

Fatty alcohols which can be used as contemplated herein include for example the unbranched C₁₄-C₅₀ fatty alcohols, in particular the C₁₄-C₃₀ fatty alcohols obtained from natural fats, oils and waxes, such as for example myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. As contemplated herein, preference is given to unbranched, saturated and unsubstituted fatty alcohols.

However, branched, saturated or unsaturated fatty alcohols can also be used as contemplated herein. Fatty alcohol cuts which are obtained in the reduction of naturally occurring fats and oils can also be used as contemplated herein.

Also suitable as lipid components are C₁₂-C₄₀ fatty acids or mixtures thereof. These include for example lauric, tridecanoic, myristic, pentadecanoic, palmitic, margaric, stearic, nonadecanoic, arachic, behenic, lignoceric, cerotic, melissic, erucic and elaeostearic acid and also substituted fatty acids, such as 12-hydroxystearic acid for example, and the amides or monoethanolamides of the fatty acids, this list being of purely exemplary and non-limiting nature. Among the fatty acids, a C₂₀-C₄₀ fatty acid mixture which is marketed under the name Performacid® 350 Acid is particularly suitable.

Waxes are understood to be natural or synthetically obtained substances having the following properties: they are of solid to brittle hard consistency, coarse to fine crystalline, transparent to opaque and melt above about 30° C. without decomposing. They are already of low viscosity just above the melting point and are non-stringy and exhibit a highly temperature-dependent consistency and solubility. Waxes which can be used as contemplated herein are for example natural plant waxes having a melting point of about 30 to about 150° C., such as for example candelilla wax, carnauba wax, Japan wax, esparto grass wax, cork wax, guaruma wax, rice oil wax, sugar cane wax, ouricury wax, montan wax, sunflower wax, fruit waxes such as orange waxes, lemon waxes, grapefruit wax, bayberry wax, and animal waxes such as for example beeswax, shellac wax, spermaceti, wool wax and uropygial fat. As contemplated herein, it may be advantageous to use hydrogenated waxes. Natural waxes which can be used as contemplated herein also include the mineral waxes, such as for example ceresin and ozokerite, or the petrochemical waxes, such as for example petrolatum, paraffin waxes and microwaxes. Chemically modified waxes, in particular the hard waxes such as for example montan ester waxes, Sasol waxes and hydrogenated jojoba waxes, can also be used as the wax component. The synthetic waxes which can be used as contemplated herein include for example wax-like polyalkylene waxes and polyethylene glycol waxes.

The wax component may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids, provided that the wax component or the entirety of the wax components melt in the range from about 30 to about 150° C. These include for example C₁₆-C₄₀ alkyl stearates, C₂₀-C₄₀ alkyl stearates (for example Kesterwachs® K82H), C₂₀-C₄₀ dialkyl esters of dimer acids, C₁₈-C₃₈ alkyl hydroxystearoyl stearates or C₂₀-C₄₀ alkyl erucates. It is also possible to use with advantage C₃₀-C₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di-(12-hydroxystearate), stearyl stearate, palmityl stearate, stearyl behenate, cetyl ester, cetearyl behenate and behenyl behenate. Silicone waxes are also advantageous where necessary.

Regardless of the type of lipid component(s) used, preference is given to hair treatment agents as contemplated herein which contain, based on their weight, about 0.01 to about 5% by weight, preferably about 0.05 to about 3% by weight, more preferably about 0.1 to about 2.5% by weight and in particular about 0.2 to about 2% by weight lipid component(s).

In preferred hair treatment agents, the lipid component(s) is/are selected from triglyceride fats, mono- and diglycerides of saturated, linear C₁₂-C₄₀ fatty acids, natural and synthetic waxes, fatty and wax alcohols, C₁₀-C₄₀ fatty acids, esters of fatty alcohols and fatty acids which are not liquid at 25° C., and also fatty acid amides and any mixtures of said substances.

Particularly preferred hair treatment agents as contemplated herein contain, based on their weight, about 0.01 to about 5% by weight, preferably about 0.05 to about 3% by weight, more preferably about 0.1 to about 2.5% by weight and in particular about 0.2 to about 2% by weight triglyceride fats and/or mono- and diglycerides of saturated, linear C₁₂-C₄₀ fatty acids and/or natural and synthetic waxes and/or fatty and wax alcohols and/or C₁₀-C₄₀ fatty acids and/or esters of fatty alcohols and fatty acids which are not liquid at 25° C. and/or fatty acid amides or any mixtures of said substances.

The hair treatment agents contain the above-described active substances preferably in a cosmetically acceptable carrier. As contemplated herein, this will be understood to mean preferably an aqueous or aqueous-alcoholic carrier.

The cosmetic carrier contains preferably at least about 50% by weight, more preferably at least about 60% by weight, particularly preferably at least about 70% by weight and more particularly preferably at least about 75% by weight water.

The cosmetic carrier may also contain about 0.01 to about 40% by weight, preferably about 0.05 to about 30% by weight and in particular about 0.1 to about 20% by weight of at least one alcohol.

Suitable alcohols are for example ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol or mixtures of said alcohols.

The water-soluble alcohols are particularly preferred. Particular preference is given to ethanol, 1,2-propylene glycol, glycerol, benzyl alcohol and mixtures of said alcohols.

For very good (scalp) skin tolerability of the hair treatment agents as contemplated herein, it is advantageous if said agents have a slightly acidic pH value.

It has been found that the agents as contemplated herein are particularly well tolerated by the skin and are particularly gentle in a pH range of from 4.2 to 5.8.

In a first preferred embodiment, the hair treatment agents as contemplated herein therefore preferably have a pH in the range from 4.2 to 5.8, more preferably from 4.25 to 5.6, particularly preferably from 4.3 to 5.5, extremely preferably from 4.35 to 5.4 and more particularly preferably from 4.4 to 5.3.

The hair treatment agents as contemplated herein may contain plant oils, plant butters and/or plant waxes. These plant oil components give the hair improved combability and manageability and increase the shine of the hair.

Suitable plant oil components include natural (plant) oils and/or butters, which usually contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

As suitable natural or plant waxes, use may preferably be made of carnauba wax, beeswax and/or candelilla wax.

Particularly preferred plant oil components are (sweet) almond oil, peach kernel oil, apricot kernel oil, amaranth seed oil, argan oil, olive oil, jojoba oil, cocoa butter and/or shea butter. Apricot kernel oil, argan oil, olive oil and/or jojoba oil are more particularly preferred.

In one preferred embodiment, the hair treatment agents as contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheatgerm oil, pumpkin seed oil, mallow oil, hazelnut oil, safflower oil, canola oil, sasanqua oil, jojoba oil, rambutan oil, cocoa butter and/or shea butter.

Within this embodiment, it is particularly preferred if the hair treatment agents as contemplated herein contain (sweet) almond oil, peach kernel oil, apricot kernel oil, amaranth seed oil, argan oil, olive oil, jojoba oil, cocoa butter and/or shea butter.

The proportion by weight of the at least one plant oil, of the plant butter and/or of the plant wax relative to the total weight of the hair treatment agents as contemplated herein is preferably about 0.02 to about 2.50% by weight, more preferably about 0.03 to about 2.00% by weight, particularly preferably about 0.04 to about 1.50% by weight and in particular about 0.05 to about 1.00% by weight.

Besides the aforementioned essential and optional constituents, the hair treatment agents as contemplated herein may in another preferred embodiment contain, in order to further increase the care properties of the agents, at least one further hair-conditioning active substance which may be selected from the group consisting of
protein hydrolysates,
vitamins,
plant extracts and/or
glycerol.

Suitable protein hydrolysates are to be understood to be product mixtures which can be obtained by acidically, basically or enzymatically catalyzed breakdown of proteins.

Protein hydrolysates of plant, animal and/or marine origin may be used.

Animal protein hydrolysates are for example elastin, collagen, keratin, silk and milk protein hydrolysates, which may also be present in the form of salts. Such products are marketed for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Preference is given to protein hydrolysates of plant origin, for example soy, almond, rice, pea, potato and wheat protein hydrolysates. Such products are available for example under the trademarks Gluadin® (Cognis), DiaMin® (Diamalt), Lexein® (Inolex) and Crotein® (Croda). Cationized protein hydrolysates can also be used, wherein the underlying protein hydrolysate can derive from animal sources, for example from collagen, milk or keratin, from plant sources, for example from wheat, maize, rice, potatoes, soy or almonds, from marine life forms, for example from fish collagen or algae, or from protein hydrolysates obtained by biotechnology. The protein hydrolysates underlying the cationic derivatives can be obtained from the corresponding proteins by means of a chemical, in particular alkaline or acid hydrolysis, an enzymatic hydrolysis and/or a combination of both types of hydrolysis. The hydrolysis of proteins generally gives rise to a protein hydrolysate having a molecular weight distribution from approximately about 100 daltons to up to several thousand daltons. Preference is given to those cationic protein hydrolysates with an underlying protein component having a molecular weight of from about 100 to about 25,000 daltons, preferably about 250 to about 5000 daltons. Cationic protein hydrolysates are also to be understood to include quaternized amino acids and mixtures thereof. The quaternization of the protein hydrolysates or amino acids is frequently performed using quaternary ammonium salts such as for example N,N-dimethyl-N-(n-alkyl)-N-(2-hydroxy-3-chloro-n-propyl) ammonium halides. The cationic protein hydrolysates can moreover also be further derivatized. Typical examples of cationic protein hydrolysates and derivatives are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) relative to the total weight of the hair treatment agents is preferably about 0.01 to about 5% by weight, more preferably about 0.025 to about 3% by weight and in particular about 0.05 to about 2% by weight.

Regardless of the source (plant, animal, marine, etc.), protein hydrolysates contain individual amino acids, oligopeptides and optionally polypeptides, depending on the degree of hydrolysis.

With particular preference, the hair treatment agents as contemplated herein contain at least one oligopeptide which has at least one amino acid sentience Glu-Glu-Glu (SEQ. ID NO: 1)

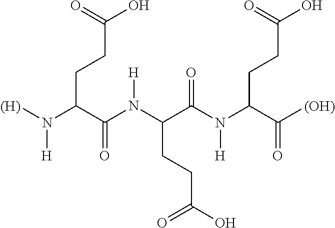

wherein the amino group may be present in free or protonated form and the carboxy groups may be present in free or deprotonated form.

Preferred hair treatment agents as contemplated herein are characterized in that they contain, based on their weight, about 0.0001 to about 10% by weight of at least one oligopeptide which has at least one amino acid sequence Glu-Glu-Glu (SEQ ID NO: 1)

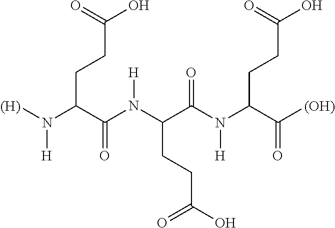

wherein the amino group may be present in free or protonated form and the carboxy groups may be present in free or deprotonated form.

In this as in all of the formulae below, the bracketed hydrogen atom of the amino group, like the bracketed hydroxyl group of the acid function, means that the groups in question may be present as such (in which case it is an oligopeptide having the respective number of amino acids as shown (in formula 3 above)) or that the amino acid sequence is present in an oligopeptide which also comprises further amino acids. Depending on where the further amino acid(s) is/are bound, the bracketed constituents in the above formula are replaced by the further amino acid residue(s).

These preferred hair treatment agents as contemplated herein contain, based on their weight, about 0.0001 to about 10% by weight of at least one oligopeptide which has at least one amino acid sequence Glu-Glu-Glu (SEQ ID NO: 1), that is to say at least three successive glutamic acids.

In the context of the present application, oligopeptides are amino acid condensation products which are linked in an acid amide-like manner by peptide bonds and which comprise at least 3 and at most 25 amino acids.

In preferred hair treatment agents as contemplated herein, the oligopeptide comprises 5 to 15 amino acids, preferably 6 to 13 amino acids, particularly preferably 7 to 12 amino acids and in particular 8, 9 or 10 amino acids.

The molecular weight of the oligopeptide contained in the agents as contemplated herein may vary depending on whether further amino acids are bound to the Glu-Glu-Glu sequence (SEQ ID NO: 1) and depending on the type of said amino acids. Preferred hair treatment agents which are used as contemplated herein are characterized in that the oligopeptide has a molecular weight of about 650 to about 3000 Da, preferably about 750 to about 2500 Da, particularly preferably about 850 to about 2000 Da and in particular about 1000 to about 1600 Da.

To sum up, preferred hair treatment agents are characterized in that the oligopeptide comprises 5 to 15 amino acids, preferably 6 to 13 amino acids, particularly preferably 7 to 12 amino acids and in particular 8, 9 or 10 amino acids and has a molecular weight of about 650 to about 3000 Da, preferably about 750 to about 2500 Da, particularly preferably about 850 to about 2000 Da and in particular about 1000 to about 1600 Da.

As can be seen from the preferred number of amino acids in the oligopeptides and the preferred molecular weight range, use is preferably made of oligopeptides which do not consist solely of the three glutamic acids but rather have further amino acids bound to said sequence (SEQ ID NO: 1). These further amino acids are preferably selected from specific amino acids, while specific other representatives are less preferred as contemplated herein.

For instance, it is preferred if the oligopeptides used in the agents as contemplated herein contain no methionine.

It is also preferred if the oligopeptides used in the agents as contemplated herein contain no cysteine and/or cystine.

It is also preferred if the oligopeptides used in the agents as contemplated herein contain no aspartic acid and/or asparagine.

It is also preferred if the oligopeptides used in the agents as contemplated herein contain no serine and/or threonine.

In contrast, it is preferred if the oligopeptides used in the agents as contemplated herein contain tyrosine.

It is also preferred if the oligopeptides used in the agents as contemplated herein contain leucine.

It is also preferred if the oligopeptides used in the agents as contemplated herein contain isoleucine.

It is also preferred if the oligopeptides used in the agents as contemplated herein contain arginine.

It is also preferred if the oligopeptides used in the agents as contemplated herein contain valine.

Particularly preferred oligopeptides and amino acid sequences contained in the preferred oligopeptides will be described below.

One particularly preferred oligopeptide additionally contains tyrosine, which is preferably bound via its acid function to the Glu-Glu-Glu sequence (SEQ ID NO: 1). Hair treatment agents which are preferred as contemplated herein are therefore characterized in that the oligopeptide contained therein has at least one amino acid sequence Tyr-Glu-Glu-Glu (SEQ ID NO: 2)

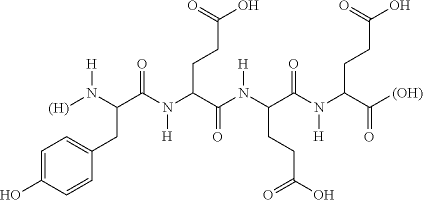

wherein the amino group may be present in free or protonated form and the carboxy groups may be present in free or deprotonated form.

Another particularly preferred oligopeptide additionally contains isoleucine, which is preferably bound via its amino function to the Glu-Glu-Glu sequence (SEQ ID NO: 1). Hair treatment agents which are preferred as contemplated herein are therefore characterized in that the oligopeptide contained therein has at least one amino acid sequence Glu-Glu-Glu-Ile (SEQ ID NO: 3)

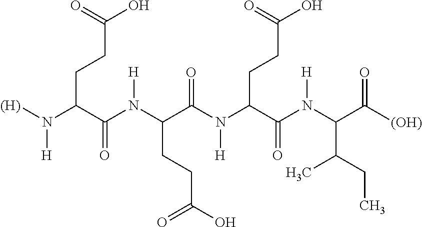

wherein the amino group may be present in free or protonated form and the carboxy groups may be present in free or deprotonated form.

As contemplated herein, preference is given to oligopeptides which contain both of the aforementioned amino acids (tyrosine and isoleucine). Particular preference is given to hair treatment agents as contemplated herein in which the oligopeptide contained in the hair treatment agent has at least one amino acid sequence Tyr-Glu-Glu-Glu-Ile (SEQ ID NO: 4)

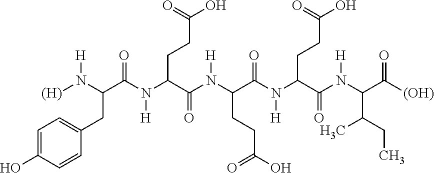

wherein the amino group may be present in free or protonated form and the carboxy groups may be present in free or deprotonated form.

Further preferred oligopeptides additionally contain arginine, which is preferably bound to isoleucine.

Particular preference is given to hair treatment agents as contemplated herein in which the oligopeptide contained in the hair treatment agent has at least one amino acid sequence Tyr-Glu-Glu-Glu-Ile-Arg (SEQ ID NO: 5)

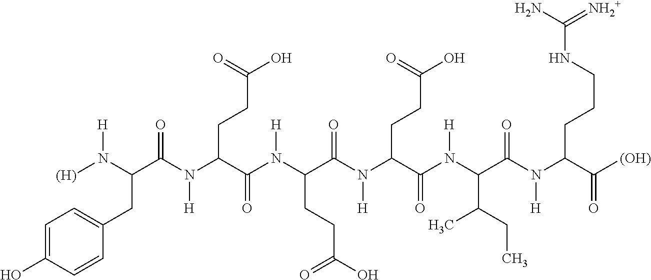

wherein the amino groups may be present in free or protonated form and the carboxy groups may be present in free or deprotonated form.

Yet further preferred oligopeptides additionally contain valine, which is preferably bound to the arginine. Hair treatment agents which are further preferred as contemplated herein are therefore characterized in that the oligopeptide contained in the hair treatment agent has at least one amino acid sequence Tyr-Glu-Glu-Glu-Ile-Arg-Val (SEQ ID NO: 6)

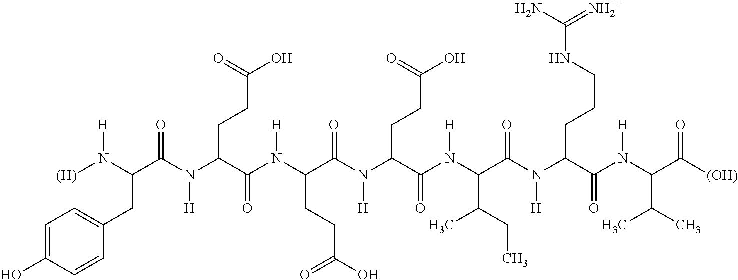

wherein the amino groups may be present in free or protonated form and the carboxy groups may be present in free or deprotonated form.

Yet further preferred oligopeptides additionally contain leucine, which is preferably bound to the valine. Hair treatment agents which are further preferred as contemplated herein are therefore characterized in that the oligopeptide contained in the hair treatment agent has at least one amino acid sequence Tyr-Glu-Glu-Glu-Ile-Arg-Val-Leu (SEQ ID NO: 7)

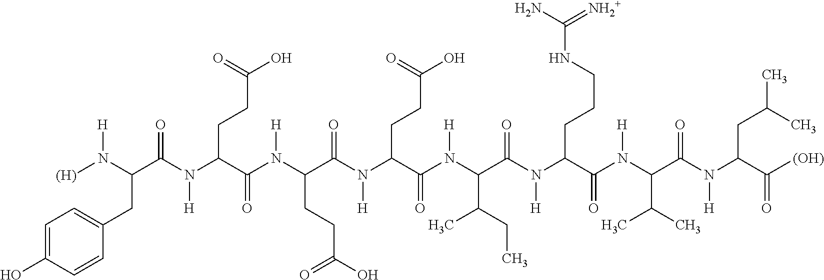

wherein the amino groups may be present in free or protonated form and the carboxy groups may be present in free or deprotonated form.

Particularly preferred oligopeptides additionally contain leucine, which is preferably bound to the tyrosine. Hair treatment agents which are further preferred as contemplated herein are therefore characterized in that the oligopeptide contained in the hair treatment agent has at least one amino acid sequence Leu-Tyr-Glu-Glu-Glu-Ile-Arg-Val-Leu (SEQ ID NO: 8)

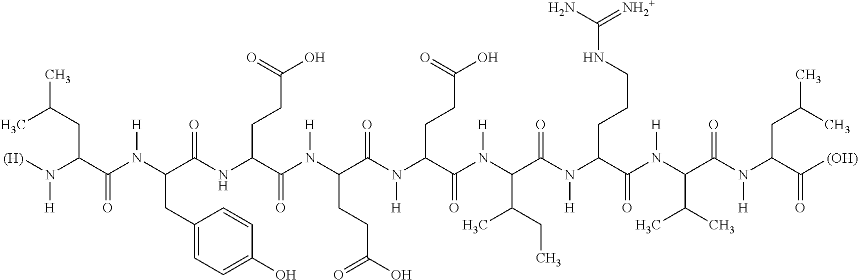

wherein the amino groups may be present in free or protonated form and the carboxy groups may be present in free or deprotonated form.

With very particular preference, agents as contemplated herein contain at least two oligopeptides which satisfy the aforementioned criteria but differ from one another. For example, preference is given to the use of hair treatment agents which contain at least two mutually different oligopeptides A and B which both contain the amino acid sequence Glu-Glu-Glu (SEQ ID NO: 1).

Such mutually different oligopeptides A and B correspond to one another in that they carry three successive Glu amino acids in their amino acid sequence but exhibit differences in the amino acids bound in front of or behind said Glu amino acids. Preference is given to mutually different peptides with partial correspondence, which correspondence may be much greater than in the aforementioned three amino acids.

Further preferred hair treatment agents are thus characterized in that the hair treatment agent contains at least two mutually different oligopeptides A and B which both contain the amino acid sequence Glu-Glu-Glu-Ile (SEQ ID NO: 3).

Preference is also given to hair treatment agents which contain at least two mutually different oligopeptides A and B which both contain the amino acid sequence Tyr-Glu-Glu-Glu (SEQ ID NO: 2).

Yet further preferred hair treatment agents are characterized in that they contain at least two mutually different oligopeptides A and B which both contain the amino acid sequence Glu-Glu-Glu-Ile-Arg (SEQ ID NO: 9).

Likewise, yet further preferred hair treatment agents are characterized in that the hair treatment agent contains at least two mutually different oligopeptides A and B which both contain the amino acid sequence Tyr-Glu-Glu-Glu-Ile (SEQ ID NO: 4).

Preferred hair treatment agents as contemplated herein are therefore characterized in that the oligopeptide has at least one amino acid sequence Tyr-Glu-Glu-Glu-Ile (SEQ ID NO: 4)

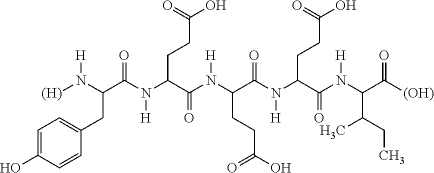

wherein the amino group may be present in free or protonated form and the carboxy groups may be present in free or deprotonated form.

Very particularly preferred hair treatment agents are characterized in that the hair treatment agent contains at least two mutually different oligopeptides A and B which both contain the amino acid sequence Glu-Glu-Glu-Ile-Arg (SEQ ID NO: 9).

Likewise, very particularly preferred hair treatment agents are characterized in that the hair treatment agent contains at least two mutually different oligopeptides A and B which both contain the amino acid sequence Tyr-Glu-Glu-Glu-Ile-Arg (SEQ ID NO: 5).

There is preferably an even greater structural correspondence in the oligopeptides. For instance, hair treatment agents containing at least two mutually different oligopeptides A and B which both contain the amino acid sequence Glu-Glu-Glu-Ile-Arg-Val (SEQ ID NO: 10) are further preferred embodiments as contemplated herein.

Hair treatment agents containing at least two mutually different oligopeptides A and B which both contain the amino acid sequence Tyr-Glu-Glu-Glu-Ile-Arg-Val (SEQ ID NO: 6) are likewise preferred embodiments.

Yet further preferred hair treatment agents as contemplated herein are characterized in that they contain at least two mutually different oligopeptides A and B which both contain the amino acid sequence Glu-Glu-Glu-Ile-Arg-Val-Leu (SEQ ID NO: 11).

Likewise, yet further preferred hair treatment agents as contemplated herein are characterized in that they contain at least two mutually different oligopeptides A and B which both contain the amino acid sequence Tyr-Glu-Glu-Glu-Ile-Arg-Val-Leu (SEQ ID NO: 7).

Preferred hair treatment agents as contemplated herein are therefore characterized in that the oligopeptide has at least one amino acid sequence Tyr-Glu-Glu-Glu-Ile-Arg-Val-Leu (SEQ ID NO: 7)

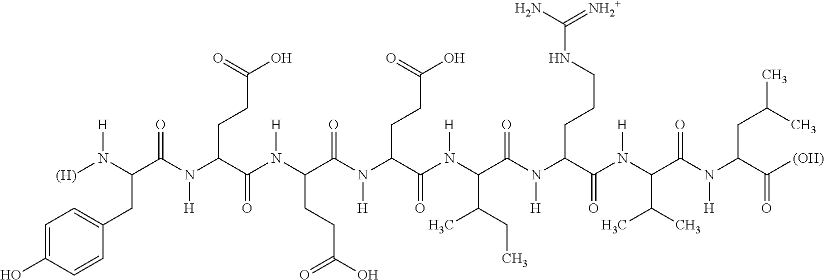

wherein the amino groups may be present in free or protonated form and the carboxy groups may be present in free or deprotonated form.

Particularly preferred hair treatment agents as contemplated herein are characterized in that they contain at least two mutually different oligopeptides A and B, wherein the oligopeptide A has the amino acid sequence Leu-Tyr-Glu-Glu-Glu-Ile-Arg-Val-Leu (SEQ ID NO: 8)

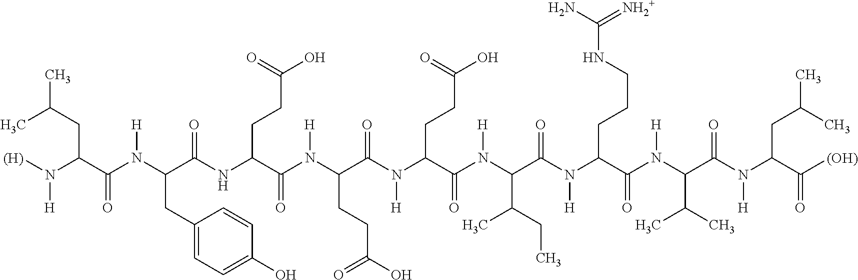

wherein the amino groups may be present in free or protonated form and the carboxy groups may be present in free or deprotonated form
and the oligopeptide B has the amino acid sequence Tyr-Glu-Glu-Glu-Ile-Arg-Val-Leu (SEQ ID NO: 7)

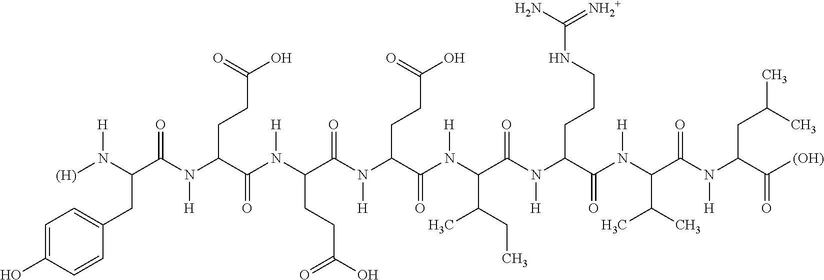

wherein the amino groups may be present in free or protonated form and the carboxy groups may be present in free or deprotonated form.

In very particularly preferred hair treatment agents of this last-mentioned embodiment, said agents contain, based on the weight of the agent, about 0.00001 to about 1% by weight oligopeptide A and about 0.00001 to about 1% by weight oligopeptide B.

In further preferred hair treatment agents of this last-mentioned embodiment, said agents contain, based on the weight of the agent, about 0.00005 to about 0.1% by weight oligopeptide A and about 0.00005 to about 0.1% by weight oligopeptide B.

In yet further preferred hair treatment agents of this last-mentioned embodiment, said agents contain, based on the weight of the agent, about 0.0001 to about 0.01% by weight oligopeptide A and about 0.0001 to about 0.001% by weight oligopeptide B.

The oligopeptides used in the context as contemplated herein, which satisfy the conditions mentioned above, can advantageously be obtained from keratin materials. It is preferred as contemplated herein that said oligopeptides are used in high proportions relative to the total keratin peptide content of the agents.

It is very particularly preferred that a highest possible proportion of all the keratin peptides contained in the agent as contemplated herein satisfies the conditions mentioned above.

Preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Glu-Glu-Glu (SEQ ID NO: 1).

Further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Glu-Glu-Glu-Ile (SEQ ID NO: 3).

Yet further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu (SEQ ID NO: 2).

Particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile (SEQ ID NO: 4).

Very particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg (SEQ ID NO: 5).

Yet further preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg-Val (SEQ ID NO: 6).

Particularly preferred hair treatment agents as contemplated herein are characterized in that at least about 0.1% by weight, preferably at least about 0.5% by weight, particularly preferably at least about 1% by weight, more preferably at least about 2.5% by weight, even more preferably at least about 5% by weight and in particular at least about 10% by weight of all the keratin peptides contained in the agent have the amino acid sequence Tyr-Glu-Glu-Glu-Ile-Arg-Val-Leu (SEQ ID NO: 7).

The conditions mentioned above relate to the total content, in the agents as contemplated herein, of peptides originating from keratin materials. In addition to the oligopeptides of keratin origin, use may of course be made of further peptides and/or protein hydrolysates, for example from other native sources. Preference is given for example to the additional use of wheat protein hydrolysates.

Suitable vitamins are to be understood to mean preferably the following vitamins, provitamins and vitamin precursors, and derivatives thereof:
Vitamin A: The group of substances designated as vitamin A includes retinol (vitamin A₁) and also 3,4-didehydroretinol (vitamin A2). β-Carotene is the provitamin of retinol. As vitamin A components, mention may be made of, for example, vitamin A acid and esters thereof, vitamin A aldehyde and vitamin A alcohol and esters thereof such as the palmitate and acetate.
Vitamin B: The vitamin B group or the vitamin B complex includes inter alia
   Vitamin B₁ (thiamine)
   Vitamin B₂ (riboflavin)
   Vitamin B₃. The compounds nicotinic acid and nicotinamide (niacinamide) are often included under this designation.
   Vitamin B₅ (pantothenic acid and panthenol). In the context of this group, use is preferably made of panthenol. Derivatives of panthenol which can be used are in particular the esters and ethers of panthenol, pantolactone and also cationically derivatized panthenols. Individual representatives are, for example, panthenol triacetate, panthenol monoethyl ether and the monoacetate thereof, as well as cationic panthenol derivatives.
   Vitamin B₆ (pyridoxine as well as pyridoxamine and pyridoxal).
Vitamin C (ascorbic acid). Use in the form of the palmitate ester, the glucosides or phosphates may be preferred. Use in combination with tocopherols may likewise be preferred.
Vitamin E (tocopherols, in particular α-tocopherol).
Vitamin F: The term “vitamin F” is usually understood to mean essential fatty acids, in particular linoleic acid, linolenic acid and arachidonic acid.
Vitamin H: The compound (3aS,4S,6aR)-2-oxohexahydrothienol[3,4-d]imidazole-4-valeric acid is designated as vitamin H, for which the trivial name biotin has become accepted.

Particular preference is given to vitamins, provitamins and vitamin precursors from groups A, B, E and H. Nicotinamide, biotin, pantolactone and/or panthenol are preferred in particular.

The proportion by weight of the vitamin(s), vitamin derivative(s) and/or vitamin precursor(s) relative to the total weight of the hair treatment agents is preferably about 0.001 to about 2% by weight, particularly preferably about 0.005 to about 1% by weight and in particular about 0.01 to about 0.5% by weight.

Suitable plant extracts are to be understood to mean extracts which can be produced from all parts of a plant. These extracts are usually produced by extraction of the entire plant. In individual cases, however, it may also be preferred to produce the extracts exclusively from blossoms and/or leaves of the plant. The extracts from green tea, oak bark, stinging nettle, witch hazel, hops, chamomile, burdock root, horsetail, whitethorn, lime blossom, lychee, almond, aloe vera, pine needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, lemon balm, restharrow, coltsfoot, marshmallow, ginseng, ginger root, Echinacea purpurea, Olea europaea, Boerhavia diffusa roots, Foeniculum vulgaris and Apium graveolens are especially suitable.

The extracts from green tea, stinging nettle, witch hazel, chamomile, aloe vera, ginseng, Echinacea purpurea, Olea europea and/or Boerhavia diffusa roots are particularly preferred for use in the compositions as contemplated herein.

As extraction agents for producing the aforementioned plant extracts, use may be made of water, alcohols and mixtures thereof. Among the alcohols, preference is given to lower alcohols such as ethanol and isopropanol, but in particular polyhydric alcohols such as ethylene glycol and propylene glycol, both as the sole extraction agent and in a mixture with water. Plant extracts based on water/propylene glycol in a ratio of 1:10 to 10:1 have proven to be particularly suitable.

The plant extracts may be used either in pure form or in dilute form. When used in dilute form, they usually contain approximately about 2 to about 80% by weight active substance, and as the solvent the extraction agent or extraction agent mixture used for the production of said plant extracts.

The plant extracts can be used in the hair treatment agents as contemplated herein (based on the total weight of the agent) preferably in an amount of about 0.01 to about 10% by weight, more preferably about 0.05 to about 7.5% by weight and in particular about 0.1 to about 5% by weight.

Glycerol may be added separately to the hair cleaning and care agents in an amount of up to about 10% by weight (based on the total weight of the agent). However, it may also be a constituent of the aforementioned aqueous-alcoholic carrier.

It has been found that the hair treatment agents as contemplated herein are also suitable for use as an antidandruff preparation.

The total weight of antidandruff agents relative to the total weight of the hair treatment agents may be preferably about 0.01 to about 10% by weight, more preferably about 0.025 to about 7.5% by weight, particularly preferably about 0.05 to about 5% by weight and in particular about 0.075 to about 3% by weight.

Suitable antidandruff active substances may be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts and/or arnica extracts.

Preference is given to climbazole, zinc pyrithione and piroctone olamine.

Further active substances, auxiliaries and additives which may preferably be contained in the hair treatment agents as contemplated herein are for example:
humectants,
perfumes,
UV filters,
thickeners such as gelatins or plant gums, for example agar-agar, guar gum, alginates, xanthan gum, gum arabic, karaya gum, locust bean gum, flaxseed gums, dextrans, cellulose derivatives, for example methylcellulose, hydroxyalkylcellulose and carboxymethylcellulose, starch fractions and derivatives such as amylose, amylopectin and dextrins, clays and phyllosilicates such as for example bentonite, or fully synthetic hydrocolloids such as for example polyvinyl alcohol, Ca, Mg or Zn soaps,
structurants such as maleic acid and lactic acid,
dimethyl isosorbide,
cyclodextrins,
active substances for improving the fiber structure, in particular mono-, di- and oligosaccharides such as for example glucose, galactose, fructose, fruit sugars and lactose,
colorants for coloring the agent,
active substances such as bisabolol and/or allantoin,
complexing agents such as EDTA, NTA, β-alaninediacetic acid and phosphonic acids,
ceramides. Ceramides are understood to be N-acylsphingosine (fatty acid amides of sphingosine) or synthetic analogs of such lipids (so-called pseudo-ceramides),
propellants such as propane-butane mixtures, N₂O, dimethyl ether, CO₂ and air,
antioxidants,
additional viscosity regulators such as salts (NaCl).

The agents as contemplated herein are preferably so-called rinse-off products, that is to say they are rinsed out of the hair again after a given leave-in time. This leave-in time is preferably less than one hour, that is to say the user preferably does not leave the products in the hair until the next time the hair is washed.

Another subject matter as contemplated herein is therefore a method for treating hair, in which an agent as contemplated herein is applied to dry or wet hair, is left thereon for a duration of from about 30 to about 300 seconds, and then is rinsed out.

The agents as contemplated herein lead to a considerably increased stability of artificial colorings against the color being washed out. Chemically colored hair can thus be washed much more frequently using the agents as contemplated herein, without this leading to undesired bleeding or fading of the color.

Another subject matter as contemplated herein is therefore the use of agents as contemplated herein to reduce the washing-out of color from chemically colored hair.

With regard to preferred embodiments of the method as contemplated herein and of the use as contemplated herein, what has been stated above in respect of the agents as contemplated herein applies mutatis mutandis.

### Examples

All figures specified in % by weight

Hair Shampoos, Series 1

| | 1 | 2 | 3 | 4 | 5 | 6 |
|---|---|---|---|---|---|---|
| Sodium laureth sulfate (AS) | 10.0 | 10.0 | 10.0 | 10.0 | 10.0 | 10.0 |
| Cocamidopropyl Betaine | 4.0 | 4.0 | 4.0 | 4.0 | 4.0 | 4.0 |
| (AS) | | | | | | |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.5 | 0.5 | 0.5 | 0.5 | 0.5 | 0.5 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Hexetidine | 0.4 | 0.4 | 0.4 | 0.4 | 0.4 | 0.4 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Colorant | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | ad 100 |

Hair Shampoos, Series 2

| | 1 | 2 | 3 | 4 | 5 | 6 |
|---|---|---|---|---|---|---|
| Sodium laureth sulfate (AS) | 7.0 | 7.0 | 7.0 | 7.0 | 7.0 | 7.0 |
| Disodium cocoamphodiacetate | 4.0 | 4.0 | 4.0 | 4.0 | 4.0 | 4.0 |
| (AS) | | | | | | |
| Coco glucoside | 3.0 | 3.0 | 3.0 | 3.0 | 3.0 | 3.0 |
| Cocamide MEA (AS) | 0.3 | 0.3 | 0.3 | 0.3 | 0.3 | 0.3 |
| Guar hydroxypropyl- | 0.1 | 0.1 | 0.1 | 0.1 | 0.1 | 0.1 |
| trimonium chloride | | | | | | |
| Panthenol | 0.2 | 0.2 | 0.2 | 0.2 | 0.2 | 0.2 |
| Jojoba seed oil | 0.05 | 0.05 | 0.05 | 0.05 | 0.05 | 0.05 |
| Wheat protein hydrolysate | 0.3 | 0.3 | 0.3 | 0.3 | 0.3 | 0.3 |
| Lactic acid | 0.4 | 0.4 | 0.4 | 0.4 | 0.4 | 0.4 |
| PEG-120 methyl glucose | 0.7 | 0.7 | 0.7 | 0.7 | 0.7 | 0.7 |
| dioleate | | | | | | |
| Hexetidine | 0.1 | 0.1 | 0.1 | 0.1 | 0.1 | 0.1 |
| Ethyl lauroyl arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Phenoxyisopropanol | — | 1.0 | 0.5 | 0.25 | 0.2 | 0.1 |
| o-Cymen-5-ol | — | 0.1 | 0.05 | 0.1 | 0.05 | 0.1 |
| o-Phenylphenol | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Colorant | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
a) at least one anionic surfactant,
b) at least one amphoteric and/or nonionic surfactant,
c) at least one cationic polysaccharide polymer,
d) hexetidine,
e) at least one organic acid, and
f) at least one lipid component.

2. The hair treatment agent according to claim 1, comprising, based on its weight, about 0.5 to about 20% by weight of a) anionic surfactant(s).

3. The hair treatment agent according to claim 1, comprising as a) alkyl (ether) sulfates of general formula R—(OCH₂—CH₂)ₙ—OSO₃X, in which R is a straight-chain or branched, saturated or unsaturated alkyl group having 8 to 24 C atoms, n is the number 0 or 1 to 12, and X is an alkali, alkaline earth, ammonium or alkanolamine ion.

4. The hair treatment agent according to claim 1, comprising, based on its weight, about 0.3 to about 10% by weight of b) amphoteric surfactant(s).

5. The hair treatment agent according to claim 1, characterized in that it contains, based on its weight, about 0.3 to about 10% by weight of b) nonionic surfactant(s).

6. The hair treatment agent according to claim 1, comprising as cationic polysaccharide polymer(s) c) at least one polymer chosen from the group consisting of cationic cellulose polymers and/or cationic guar derivatives.

7. The hair treatment agent according to claim 1, comprising, based on its weight, about 0.001 to about 0.3% by weight of d) hexetidine.

8. The hair treatment agent according to claim 1, comprising as e) organic acid(s) chosen from the group of formic acid, oxalic acid, maleic acid, lactic acid, tartaric acid or citric acid.

9. The hair treatment agent according to claim 1, comprising, based on its weight, about 0.01 to about 5% by weight of f) lipid component(s).

10. The hair treatment agent according to claim 1, wherein the f) lipid component(s) is/are selected from triglyceride fats, mono- and diglycerides of saturated, linear C12-C40 fatty acids, natural and synthetic waxes, fatty and wax alcohols, C10-C40 fatty acids, esters of fatty alcohols and fatty acids which are not liquid at 25° C., and fatty acid amides and any mixtures of said substances.

11. The hair treatment agent according to claim 3, comprising as a), alkyl (ether) sulfates of general formula R—(OCH₂—CH₂)ₙ—OSO₃X, in which R is a straight-chain or branched, saturated or unsaturated alkyl group having 8 to 24 C atoms, n is 1 to 12, and X is an alkali, alkaline earth, ammonium or alkanolamine ion.

12. The hair treatment agent according to claim 3, comprising, based on its weight, 0.5 to 20% by weight of the alkyl (ether) sulfates.

13. The hair treatment agent according to claim 6, comprising as cationic polysaccharide polymer(s) c), based on the weight of the agent, about 0.01 to about 3% by weight, of the at least one polymer chosen from the group consisting of cationic cellulose polymers and/or cationic guar derivatives.

14. The hair treatment agent according to claim 8, comprising, based on its weight, about 0.001 to about 10% by weight, organic acid(s) chosen from the group of formic acid, oxalic acid, maleic acid, lactic acid, tartaric acid or citric acid.

15. The hair treatment agent according to claim 1, comprising, based on its weight:
about 0.5 to about 20% by weight of a) anionic surfactant(s);
about 0.3 to about 10% by weight of b) at least one amphoteric and/or nonionic surfactant;
about 0.01 to about 3% by weight, of the c) at least one cationic polysaccharide polymer;
about 0.001 to about 0.3% by weight of d) hexetidine;
about 0.001 to about 10% by weight of e) organic acid(s); and
about 0.01 to about 5% by weight of f) lipid component(s).

16. The hair treatment agent according to claim 1, comprising:
as a) alkyl (ether) sulfates of general formula R—(OCH₂—CH₂)ₙ—OSO₃X, in which R is a straight-chain or branched, saturated or unsaturated alkyl group having 8 to 24 C atoms, n is the number 0 or 1 to 12, and X is an alkali, alkaline earth, ammonium or alkanolamine ion;
as c) cationic polysaccharide polymer(s) at least one polymer chosen from the group consisting of cationic cellulose polymers and/or cationic guar derivatives;
as e) organic acid(s) chosen from the group of formic acid, oxalic acid, maleic acid, lactic acid, tartaric acid or citric acid; and
as f) lipid component(s) selected from triglyceride fats, mono- and diglycerides of saturated, linear C12-C40 fatty acids, natural and synthetic waxes, fatty and wax alcohols, C10-C40 fatty acids, esters of fatty alcohols and fatty acids which are not liquid at 25° C., and fatty acid amides and any mixtures of said substances.

17. A method for treating hair, comprising:
applying a hair treatment agent to dry or moist hair, wherein the hair treatment agent comprises:
a) at least one anionic surfactant,
b) at least one amphoteric and/or nonionic surfactant,
c) at least one cationic polysaccharide polymer,
d) hexetidine,
e) at least one organic acid, and
f) at least one lipid component,
where the hair treatment is agent is left on the hair for a period of about 30 to about 300 seconds and then rinsed off.
